(19) United States
(12) Patent Application Publication
(10) Pub. No.: US 2016/0171154 A1
(43) Pub. Date: Jun. 16, 2016

(54) ENHANCED DETECTION OF NON-SOMATIC CIRCULATING NUCLEIC ACIDS

(71) Applicant: Frederick S. Wreford, Cadillac, MI (US)

(72) Inventor: Frederick S. Wreford, Cadillac, MI (US)

(21) Appl. No.: 15/053,319

(22) Filed: Feb. 25, 2016

Related U.S. Application Data
(62) Division of application No. 13/621,414, filed on Sep. 17, 2012.

(51) Int. Cl. G06F 19/22; C12Q 1/68

(52) U.S. Cl. CPC G06F 19/22; C12Q 1/6883

(57) ABSTRACT

Methods, equipment and software for the detection of abnormal nucleic acid or fetal nucleic acid sequences circulating in the subjects blood or other body fluids. A scan of a subject's circulating nucleic acid (CNA) is compared to the CNA Data On Normal Healthy Population or to a scan of the subject's own Somatic Nucleic Acid (SNA). Abnormal peaks are identified by subtracting out the “normal” or somatic peaks. The resultant anomalous peaks are compared to anomalous CNA data to identify the abnormalities, or the anomalous peaks of the subjects CNA are isolated, amplified, and sequenced or probed to determine the nature of the anomaly.

### CLAIM OF PRIORITY

This is a divisional of application Ser. No. 13/621,414, filed Sep. 17, 2012, and which claims the benefit of U.S. Provisional patent application Ser. No. 61/535,762, filed Sep. 16, 2011.

### FIELD OF THE INVENTION

The present invention is related to the use of biomarkers as indicators of disease, genetic disorders, infection, and other disorders and abnormalities.

### FEDERALLY SPONSORED RESEARCH/DEVELOPMENT

No federally sponsored research/development was involved in the development of this patent application.

### BACKGROUND OF THE INVENTION

Most of the DNA and RNA in the body is located within cells. However, a small amount of nucleic acids can also be found circulating freely in the blood. The term “Circulating Nucleic Acids” (CNA) refers to these segments of DNA or RNA found in the bloodstream, plasma, serum or other body fluid.

There is great interest in these CNAs as possible markers for disease and genetic disorders. As stated in an article by Y. M. DENNIS LO entitled CIRCULATING NUCLEIC ACIDS IN PLASMA OR SERUM, Annals of the New York Academy of Sciences 2001, Volume 945 Issue II, Pages 1-7:

“The recent interest in nucleic acids in plasma and serum has opened up numerous new areas of investigation and new possibilities for molecular diagnosis. In oncology, tumor-derived genetic changes, epigenetic alterations, and viral nucleic acids have been found in the plasma/serum of cancer patients. These findings have important implications for the detection, monitoring, and prognostication of many types of malignancies. In prenatal diagnosis, the discovery of fetal DNA in maternal plasma and serum has provided a noninvasive source of fetal genetic material for analysis. This development has important implications for the realization of noninvasive prenatal diagnosis and has provided new methods for the monitoring, of pregnancy-associated disorders. Plasma DNA technology has also found recent. applications in the fields of organ transplantation, post-trauma monitoring, and infectious agent detection. Future areas of study include circulating RNA in plasma and the elucidation of the biology of release, clearance, and possible functionality of plasma nucleic acids.”

Unfortunately, most the CNAs are somatic in origin, resulting for example from apoptosis of healthy somatic cells, or spontaneous release of newly synthesized DNA or RNA. Only low levels of DNA are yielded from the presence and breakdown of pathogens, or the presence of fetal DNA in maternal plasma. This makes detection of potential biomarkers difficult.

### BRIEF SUMMARY OF THE INVENTION

The present invention comprises methods, equipment and software for the detection of abnormal nucleic acid or fetal nucleic acid sequences circulating in the subject's blood or other body fluids. A scan of a subject's circulating nucleic acid (CNA) is compared to the CNA Data On Normal Healthy Population or to a scan of the subject's own Somatic Nucleic Acid (SNA). Abnormal peaks are identified by subtracting out the “normal” or somatic peaks. The resultant anomalous peaks are compared to anomalous CNA data to identify the abnormalities, or the anomalous peaks of the subject's CNA are isolated, amplified, and sequenced or probed to determine the nature of the anomaly.

A diagnosis or the identification of a new marker can then be made based on the results obtained. These and other objects and advantages will be more fully understood and appreciated by reference to the description of the preferred embodiments and to the drawings.

### DESCRIPTION OF THE DRAWINGS

FIG. 1 is a table outlining preferred embodiment methods for practicing the invention.

FIG. 2 is an electronic scan of a circulating nucleic acid analysis;

FIG. 3 is an electronic scan of a healthy somatic nucleic acid analysis;

FIG. 4 is the scan resulting from the subtraction of the healthy somatic control scan from the CNA scan.

### DESCRIPTION OF THE PREFERRED EMBODIMENTS

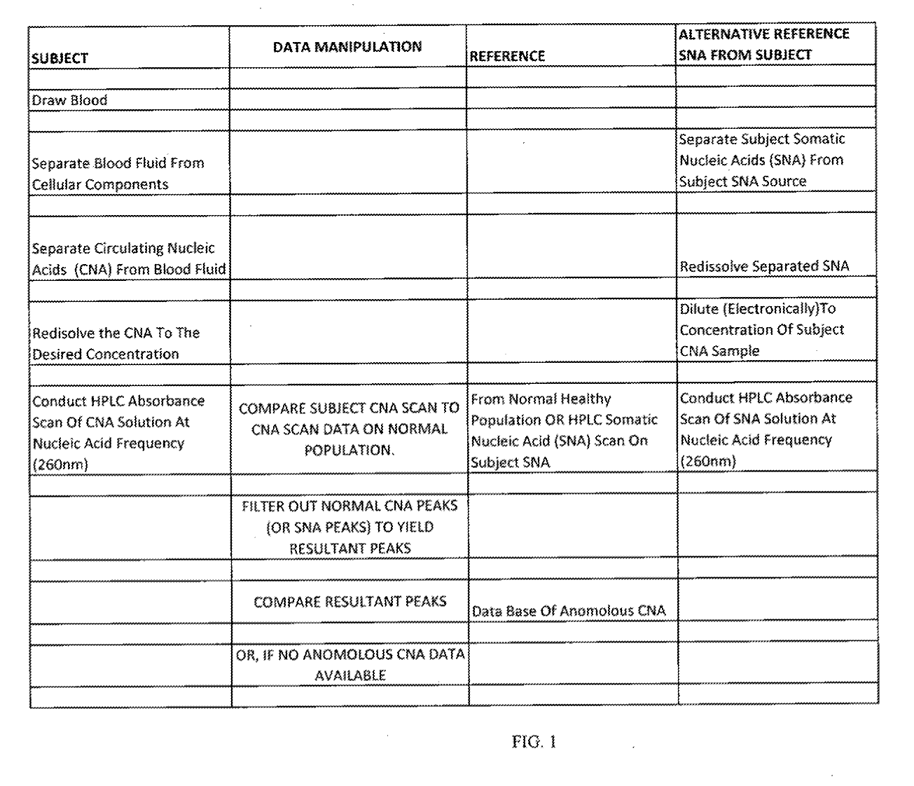

The preferred embodiment comprises integrated equipment and software for performing the preferred embodiment methods described herein. However, the methods described can be performed using known techniques and equipment. FIG. 1 comprises a table summarizing the preferred embodiment methods. Column 1 entitled “Subject” identifies the operations performed on the subject, that is the person whose circulating nucleic acids are being evaluated. Column 2 entitled “Data Manipulation” identifies what is done with the data by the software or by manual activity. Column 3 entitled “Reference” identifies the reference material utilized, and column 4 entitled “Alternative Reference” identifies the steps necessary to use as a reference the somatic nucleic acids originating in the subject himself or herself.

A peripheral blood or body fluid sample is drawn from the individual to be tested. The fluid portion of the sample is separated from the cellular components of the blood or other body fluid sample using standard laboratory techniques. In the case of blood, this is typically accomplished by centrifugation, which results in the red blood cells settling at the bottom of the sample, and the clear fluid containing circulating nucleic acids being located at the top. A thin layer in between is the so-called “buffy coat,” which contains most of the white blood cells and platelets.

Further separation techniques can be used to separate the circulating nucleic acids from proteins or other material which may be circulating in the blood fluid. The separated nucleic acids are then redissolved to a desired concentration, which should be consistent with the concentration of nucleic acids in the reference material to which the redissolved CNA sample is to be compared. To the extent the concentrations are not comparable, the concentration of the subject specimen is adjusted electronically by the software controlling the absorbance comparison of the subject sample to the reference.

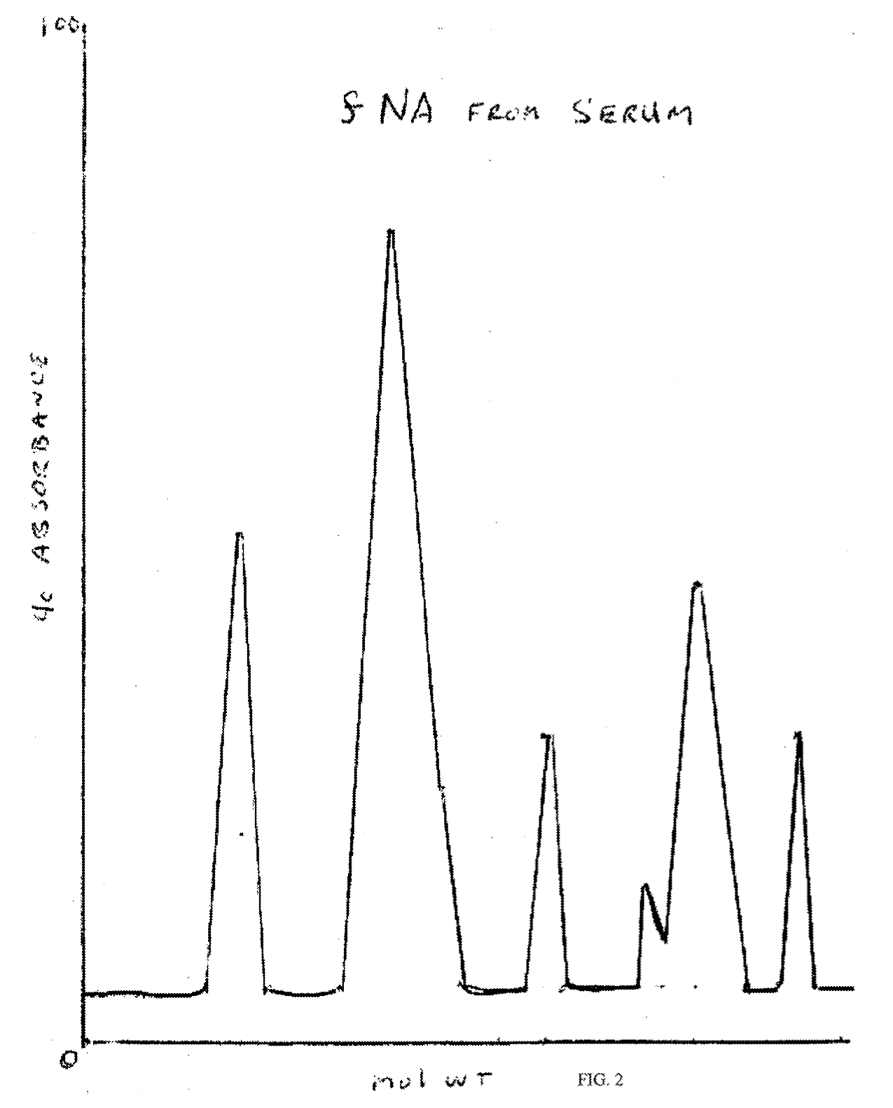

The next step is to conduct a high pressure liquid chromatography absorbance scan of the circulating nucleic acid solution at an appropriate absorbance frequency. Typically, nucleic acids absorb at 260 nanometers. The resulting scan will show peaks, representing nucleic acid fragments of varying sizes coming through the HPLC apparatus at different times. The smallest fragments travel fastest and come through first, and the largest fragments of course travel more slowly and come through later. The solution is scanned as it passes out of the separation column or medium. The resulting scan is exemplified in FIG. 2.

The resulting absorbance scan is then compared to reference data on the circulating nucleic acids found in normal healthy populations, or to a somatic nucleic acid scan conducted on the subject's own somatic nucleic acid. The data from a normal, healthy population would be presented in the form of an HPLC separation scan, with different peaks representing different sized nucleic acid segments. Such scans comprise absorbance on the Y axis and the passage of time on the X axis, The software provided would electronically adjust the concentration in either the normal population CNA scan or the subject CNA scan, to simulate comparable overall concentration, so that corresponding peaks would be of comparable size. The rate at which the HPLC separation scan is conducted on the subject sample would have to be comparable to the rate at which the reference data has been scanned, or if not, a sampling rate adjustment must be made by the software, so that the scan rates appear comparable.

In the event CNA data from a normal healthy population is not available, or as an additional comparison, a scan can be taken of somatic nucleic acid obtained from the subject being evaluated. Somatic nucleic add will come from normal healthy cells of the subject. These “somatic cells” can be any nucleated cell, as for example lymphocytes, tissue samples, etc.

One convenient source would be the lymphocytes taken from the “buffy coat” in the separated blood samples of the subject Excess plasma would be washed out of the buffy coat using an isotonic salt solution. The white blood cells would then be lysed to release the somatic DNA or RNA from those cells. The buffy coat approach cannot be used if the patient has lymphoma or leukemia, and an alternative source of somatic cells would have to be selected.

The somatic nucleic acid would then be separated from the other components of the lysed cells using conventional laboratory techniques. The separated somatic nucleic acid would be redissolved to a concentration comparable to that used for the CNA solution. Alternatively, the overall concentration of nucleic acid would be adjusted electronically by the software so that the overall SNA and CNA concentrations would be comparable.

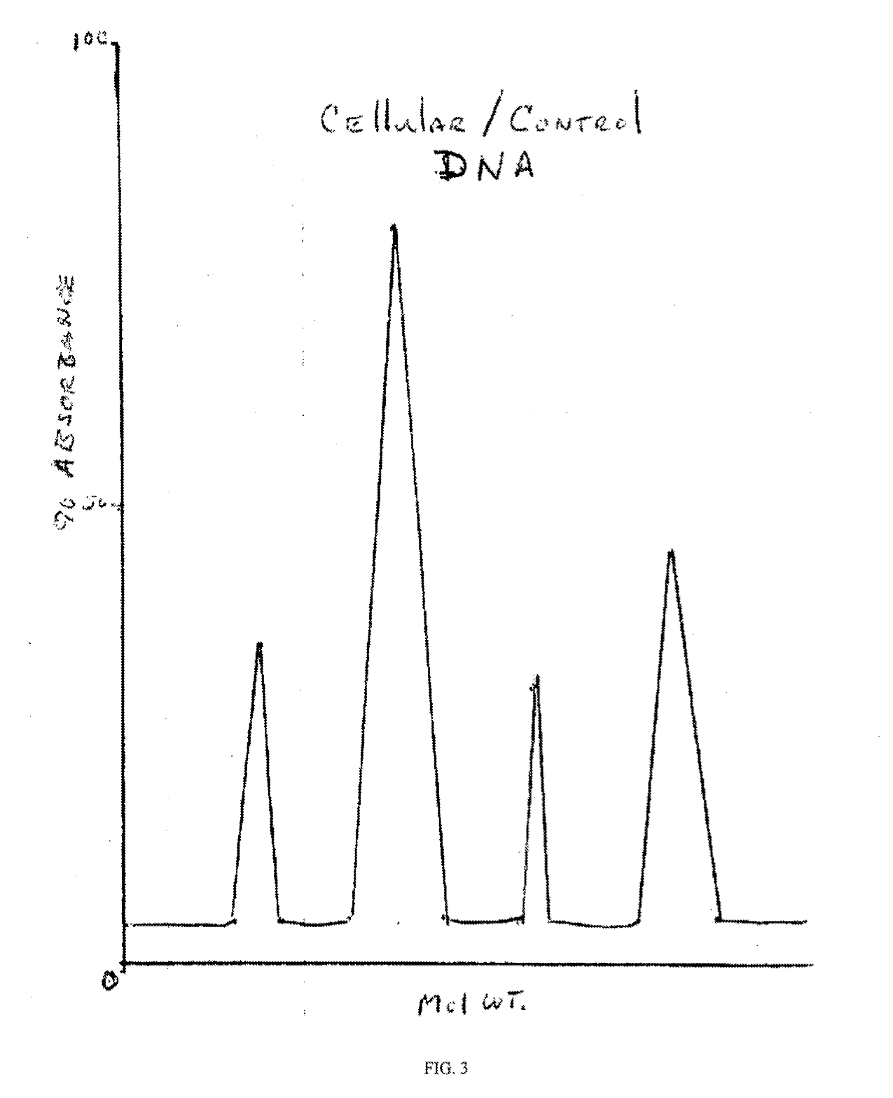

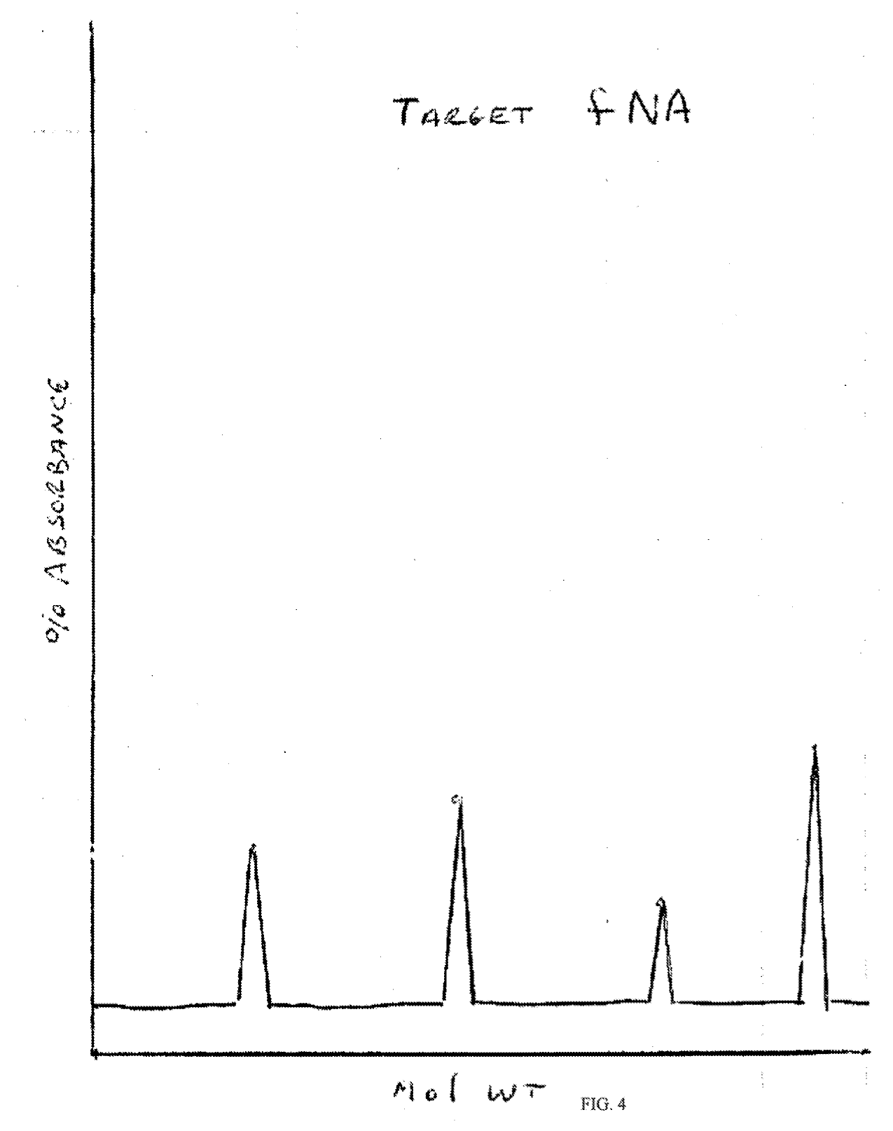

FIG. 3 shows a representative composite HPLC separation scan for a normal healthy population, or an individual scan of a subjects somatic nucleic acids. The software compares the scans of FIGS. 2 and 3, and filters out the CNA peaks appearing in a normal healthy population (or filters out the somatic nucleic acid peaks), yielding “resultant peaks” which comprise anomalous CNA segments circulating in the subject's blood or other bodily fluids. (See FIG. 4)

The resultant peaks (FIG. 4) are then compared to a database of anomalous CNA, once such a database is available. The anomalous CNA data would identify various anomalous peaks as markers for particular diseases, particular genetic disorders, or markers as to the fact that the subject is pregnant. To the extent that no anomalous CNA data is available for one or more of the anomalous peaks, or to the extent that the purpose of the procedure is to contribute to such a database, an HPLC separation of the CNA located at resultant peaks is conducted. The software coordinates resultant peaks with the HPLC separation so that CNA coming off the separation column at desired peaks is separated. The separated nucleic acid samples are then amplified using PCR technology and equipment. The resulting nucleic acid segments are sequenced or identified using gene probes, and are compared to a database of disease or disorder nucleic acid markers. Alternatively, the information obtained is used to associate particular nucleic acid segments with particular diseases, disorders or conditions.

The results provide detection of additional or deleted nucleic acids sequences in the individual's circulation from fetal, malignant or other abnormal tissues. The process, apparatus and software of the present invention provides a powerful tool for diagnosing a patient on the basis of circulating nucleic acid segments comprising known markers for particular diseases, genetic disorders or conditions such as pregnancy of the subject. Alternatively, the method, apparatus and software comprise a potent tool for researching anomalous circulating nucleic acid segments and identifying particular disorders, diseases or conditions associated with the anomalous markers.

The invention claimed is:

1. A method for the detection of abnormal nucleic acid or fetal nucleic acid sequences circulating in the subject's blood or other body fluids, comprising:
making a scan of a subject's circulating nucleic acid (CNA);
making a scan of the subject's own Somatic Nucleic Acid (SNA);
comparing the CNA scan to the SNA scan;
subtracting the peaks of the SNA scan from the CNA scan;
analyzing the resultant anomalous peaks to determine their nature.

2. The method of claim 1 wherein said analysis of said resultant anomalous peaks comprises comparing them to anomalous CNA data to identify the abnormalities.

3. The method of claim 1 wherein said analysis of said resultant anomalous peaks comprises, isolating, amplifying and sequencing or probing said anomalous peaks to determine the nature of the anomaly.

4. The method of claim 1 wherein said scan is a high pressure liquid chromatography absorbance scan of the circulating nucleic acid solution at an appropriate absorbance frequency.

5. The method of claim 4 in which said scan is conducted at about 260 nanometers.

6. The method of claim 1 in which making a scan of the subject's CNA includes:
drawing a peripheral blood or body fluid sample from the subject;
separating the fluid portion of the sample from the cellular and other protein components of the sample; and
conducting said scan on said separated fluid portion.

7. The method of claim 6 in which making a scan of the subject's CNA further includes:
separating the circulating nucleic acids from said fluid portion and re-dissolving them to a desired concentration, which is consistent with the concentration of nucleic acids in said SNA scan material, to which the redissolved CNA sample is to be compared.

8. The method of claim 6 in which the concentrations of the CNA and SNA in their respective fluids are adjusted electronically by machine readable media controlling the absorbance comparison of the two samples.

9. The method of claim 1 in which making a scan of the subject's SNA includes: sampling the subject's normal healthy nucleated “somatic cells;”
lysing said cells to release the somatic DNA or RNA from said cells;
separating said somatic nucleic acid from the other components of said lysed cells;
re-dissolving said somatic nucleic acid; and
conducting said scan on the resulting somatic nucleic acid sample.

10. The method of claim 9 in which said somatic nucleic acid is re-dissolved to a concentration comparable to that used for the CNA solution.

11. The method of claim 9 in which the concentrations of the CNA and SNA in their respective fluids are adjusted electronically by machine readable media controlling the absorbance comparison of the two samples.

12. The method of claim 9 in which normal healthy nucleated “somatic cells” comprise:
lymphocytes taken from the “buff coat” in a separated blood sample of the subject.

13. Apparatus including machine readable media to facilitate the detection of abnormal nucleic acid or fetal nucleic acid sequences circulating in a subject's blood or other body fluids, comprising:
a routine for recording a scan of a subject's circulating nucleic acid (CNA);
a routine for recording a scan of the subject's own Somatic Nucleic Acid (SNA);
a comparator for comparing the CNA scan to the SNA scan;
a routine subtracting the peaks of the SNA scan from the CNA scan;
a routine for presenting the resultant anomalous peaks for further analysis.

14. The apparatus of claim 13 in which said machine readable media includes anomalous CNA data, and a routine for comparing said anomalous peaks to said anomalous CNA data to identify the source of the abnormalities.

15. The apparatus of claim 13 which isolates, amplifies and sequences or probes said resultant anomalous peaks, and said machine readable media includes a routine for controlling and reporting the results of said isolation, amplification and sequencing steps.

16. Apparatus including machine readable media to facilitate the detection of abnormal nucleic acid or fetal nucleic acid sequences circulating in a subject's blood or other body fluids, comprising:
a routine for recording a scan of a subject's circulating nucleic acid (CNA);
a routine for storing similarly scanned CNA Data On Normal Healthy Population (NHP CNA Data);
a comparator for comparing said subject's CNA scan to said similarly scanned CNA Data On Normal Healthy Population (NHP CNA Data);
a routine subtracting the peaks of the NHP CNA Data from the CNA scan;
a routine for presenting the resultant anomalous peaks for further analysis.

17. The apparatus of claim 16 in which said machine readable media includes a routine for storing anomalous CNA data, and a routine for comparing said anomalous peaks to said anomalous CNA data to identify the source of the abnormalities.

18. The apparatus of claim 16 which isolates, amplifies and sequences or probes said resultant anomalous peaks, and said machine readable media includes a routine for controlling and reporting the results of said isolation, amplification and sequencing steps.
